(19) United States
(12) Patent Application Publication
(10) Pub. No.: US 2011/0152712 A1
(43) Pub. Date: Jun. 23, 2011

(54) Impedance Measurement Tissue Identification in Blood Vessels

(76) Inventors: Hong Cao, Savage, MN (US); Christopher D. Minar, New Prague, MN (US); Pete Skujins, Brooklyn Park, MN (US); Alan D. Eskuri, Hanover, MN (US)

(21) Appl. No.: 12/973,889

(22) Filed: Dec. 20, 2010

Related U.S. Application Data
(60) Provisional application No. 61/288,418, filed on Dec. 21, 2009.

(51) Int. Cl. A61B 5/053; A61B 18/14

(52) U.S. Cl. 600/547; 606/33

(57) ABSTRACT

A system for radio frequency (RF) wire guidance during RF occlusion canalization including a RF wire configured to travel through a blood vessel, an impedance measure unit configured to measure an electrical impedance between the RF wire and a reference electrode, a RF power source, an activation controller configured to connect the RF power source to the RF wire when the impedance measure unit indicates that the RF wire is contacting an occlusion material, a RF wire steering system; and a controller configured to activate the steering system to maintain the RF wire within the occlusion material based on impedance measured by the impedance measure unit.

### CROSS-REFERENCE TO RELATED APPLICATIONS

This application claims the benefit of U.S. Provisional Application No. 61/288,418, filed on Dec. 21, 2009. The entire disclosure(s) of (each of) the above application(s) is (are) incorporated herein by reference.

### FIELD

The present disclosure relates to tissue identification, and in particular, to tissue identification in blood vessels using impedance measurement.

### SUMMARY

This section provides a general summary of the disclosure, and is not a comprehensive disclosure of its full scope or all of its features.

According to one aspect of the present disclosure, a method is provided for identifying, or at least distinguishing, different kinds of tissues, such as the different types of tissues encountered in blood vessels. In one embodiment, an electrode catheter or other medical device is navigated through the body, preferably a body lumen or cavity, such as a blood vessel. The electrode contacts tissue and measures its impedance, an absolute impedance measurement, or a relative impedance measurement can be used to determine the type of tissue adjacent the electrode, or at least to distinguish between different tissues. This acquired impedance information can be used to create a map and/or to guide further movement of the catheter, for example directing the catheter away from healthy tissue and toward atheromotous material.

According to another aspect, a device, such as an electrode catheter, is provided that can be navigated through a blood vessel in the vicinity of a partial or total vascular occlusion. One or more electrodes on the electrode catheter contacts material in the blood vessel, and the impedance of the material in the vicinity of the electrode can be measured between the electrode on the catheter, and a reference electrode which may be carried on the catheter, but is preferably separate. By moving the catheter or using different electrodes on the catheter, the impedance of the tissue at a different location in the vessel can be determined. The absolute impedance measurement at a location can be used to determine tissue type, and thereafter, determine whether or not the device should be moved. For example, if the absolute impedance measurement indicates that the electrode is in contact with healthy tissue, the catheter could be moved, but if the absolute impedance measurement indicates that the electrode is in contact with athromotous material, the catheter could be left in place to treat or remove the athromotous material, for example by ablation. Similar, a difference in impedance measurement could be used to determine tissue type, and thereafter, determine whether the catheter should be moved, For example, if the difference in impedance measurement exceeds a certain threshold, it indicates that the electrode has moved from healthy tissue to athromotous material (or vice versa). If the change in impedance measurement indicates that the electrode is in contact with athromotous material, the catheter could be left in place to treat or remove the athromotous material, for example by ablation. If the change in impedance measurement indicates that the electrode is in contact with healthy material, the catheter could be moved to a new location.

According to another aspect of the present disclosure, a system and method are provided for radio frequency (RF) wire guidance during RF occlusion canalization. The system can include an RF wire configured to be navigated through a body lumen or cavity, such as a blood vessel, an impedance measure unit configured to measure an electrical impedance between the RF wire and a reference electrode, an RF power source, an activation controller configured to connect the RF power source to the RF wire when the impedance measure unit indicates that the RF wire is contacting an occlusion material. The RF wire includes a steering system, which can be a mechanical, magnetic, electrostrictive, pneumatic, or hydraulic system for remotely changing the shape, configuration, or orientation of the distal end portion of the RF wire, and a controller configured to activate the steering system in response to user commands, or to automatically generated commands determined in part from the impedance measured by the wire. By moving the RF wire, the impedance of the tissue at different locations in the vessel can be determined. The absolute impedance measurement at a location can be used to determine tissue type, and thereafter, determine whether or not the RF wire should be steered to a new location. For example, if the absolute impedance measurement indicates that the RF wire is in contact with healthy tissue, the steering system could be operated to move the RF wire, but if the absolute impedance measurement indicates that the RF wire is in contact with athromotous material, the RF wire could be left in place to treat or remove the athromotous material, for example by ablation. Similarly, a difference in impedance measurement could be used to determine tissue type, and thereafter, determine whether the RF wire should be moved. For example, if the difference in impedance measurement exceeds a certain threshold, it indicates that the electrode has moved from healthy tissue to athromotous material (or vice versa). If the change in impedance measurement indicates that the RF wire is in contact with athromotous material, the RF wire could be left in place to treat or remove the athromotous material, for example by ablation. If the change in impedance measurement indicates that the electrode is in contact with healthy material, the RF wire could be moved to a new location by operating the steering system. Of course, trends or gradients of increasing or decreasing impedance could be tracked, and used to determine new locations or directions for controlling the steering system.

According to yet another aspect of the present disclosure, a method of characterizing tissue within a body lumen or cavity, such as a blood vessel is provided which includes contacting a first blood vessel site with an electrode on an elongate medical device, such as a wire, catheter, or endoscope, and measuring the impedance between the vascular electrode and a reference electrode (which may also be carried on the elongate device or provided separately). The measured impedance can be used to determine the nature of the tissue in contact with the electrode, or the device can be moved to a second location, and the impedance measured at the second location and the change in impedance used to determine the nature of the tissue in contact with the electrode. By successively moving the device and measuring impedance, a map of location and impedance, or map of location and tissue type can be created, and preferably, graphically displayed so that a map of atheramatous material can be created for future use in planning and carrying out treatment.

According to yet another aspect of the present disclosure, a system comprising an elongate medical device, such as an endoscope, catheter or wire including an electrode, an impedance measuring unit connected to the vascular electrode. The system may also include a steering system, which can be a mechanical, magnetic, electrostrictive, pneumatic, or hydraulic system for remotely changing the shape, configuration, or orientation of the distal end portion of the RF wire. The steering system may be manually operated, but preferably, a controller is provided for automating the operation of the steering system. The system preferably also includes a remote advancement system configured to advance or retract the medical device along a longitudinal axis of a blood vessel. The remote advancement system may be manually operated, but preferably a controller is provided for automating the operation of the remote advancement system.

A user can operate the steering system and the remote advancement system to move the medical device through a portion of a blood vessel. Alternatively, a controller can automatically operate the steering system and the remote advancement system. The controller is configured to actuate the vascular remote advancement system and the steering system to contact the vascular electrode to a plurality of blood vessel sites and measure the impedance of the plurality of blood vessel sites with the impedance measure unit. The measured impedances can be used to determine the nature of the tissue at each location, or the differences in measured impedances can be used to determine the nature of the tissue at each location. By successively moving the device and measuring impedance, a map of location and impedance, or a map of location and tissue type can be created, and preferably, graphically displayed so that a map of atheramatous material can be created for future use in planning and carrying out treatment.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a table of electrical conductivity and relative permittivity of tissues, according to one example embodiment of the present disclosure;

FIG. 2 is a circuit diagram of an impedance measuring circuit, according to one example embodiment of the present disclosure;

FIG. 3 is a table of material estimate based on complex impedance, according to one example embodiment of the present disclosure;

FIG. 4 is a circuit diagram of another impedance measuring circuit, according to one example embodiment of the present disclosure;

FIG. 5 is a circuit diagram of another impedance measuring circuit, according to one example embodiment of the present disclosure;

FIG. 6 is a block diagram of a system for characterizing tissue within a blood vessel, according to one example embodiment of the present disclosure; and

FIG. 7 is a block diagram of a system for RF occlusion canalization, according to one example embodiment of the present disclosure.

Corresponding reference numerals indicate corresponding parts throughout the several views of the drawings.

### DETAILED DESCRIPTION

Example embodiments will now be described more fully with reference to the accompanying drawings.

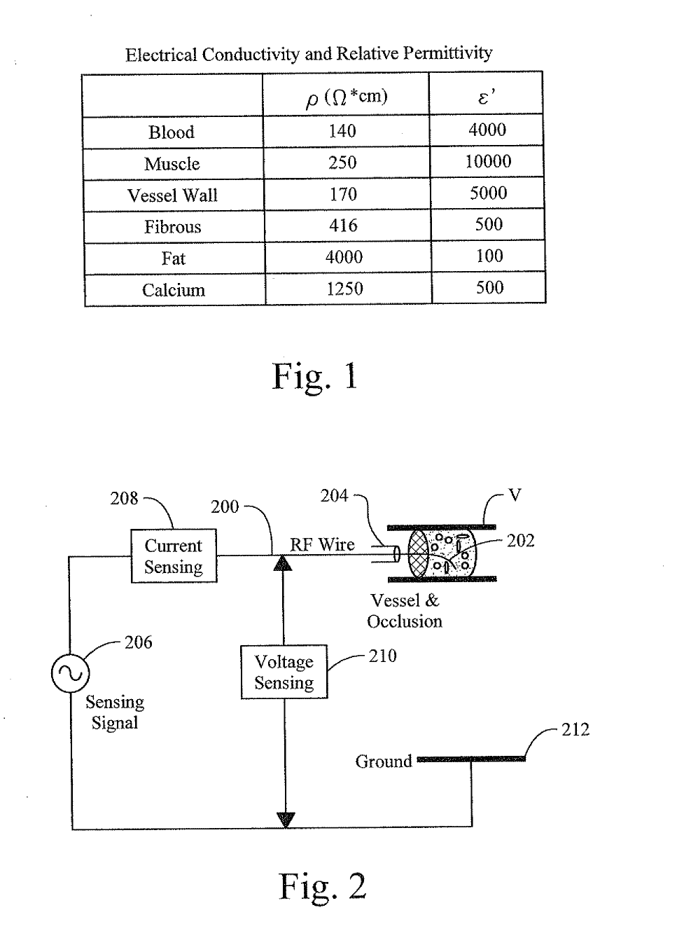

The ability of a material to conduct electrical current is based in part on its composition. Normal animal tissue and blood comprise live cells that conduct current relatively well. Atheromatous plaques typically comprise macrophage cells, cell debris, lipids (cholesterol and fatty acids), calcium, and fibrous connective tissue. For the most part, atheromatous plaques do not comprise live cells and do not have a cell membrane. Thus, atheromatous plaques typically have a higher resistivity (lower conduction) and lower relative permittivity (lower capacitance) than healthy tissue, as shown in FIG. 1. Based upon the electrical conduction or impedance, or upon changes in the electrical conduction or impedance, one can differentiate the tissue type, such as blood, muscle, vessel, or plaque using an electrode, such as, for example, an electrode carried on the distal end of an elongate medical device, such as an endoscope, catheter, or guidewire.

Such electrodes often can be used both for measuring electrical activity in tissue and applying ablative energy to tissue. In some configurations, a single electrode is provided on the elongate medical device, and a second electrode is disposed elsewhere on the subject's body. In other embodiments, multiple electrodes can be provided on the same device, such that the measurement and application of energy can be made between a pair of electrodes. When there are multiple pairs of electrodes, measurements at multiple locations, and/or the application of energy at multiple locations can be accomplished without moving the elongate medical device that is carrying the electrodes. On guidewires and other similar wire-based devices, for example, a 0.018″ RF wire, the electrode may be a conductive portion of a wire that can be used to measure impedance and also to apply radio frequency (RF) energy to tissue to vaporize the tissue, such as a 0.018″ RF wire.

The membrane of the living cell typically acts as a barrier at low frequency. As electrical frequency increases, the membrane typically behaves as a capacitor and allows current to pass through. Consequently, the impedance decreases dramatically. This phenomenon is defined as β dispersion and usually occurs at frequencies between tens of kilohertz and hundreds of kilohertz. The measurement frequency is preferably between 10 kHz and 500 kHz.

Impedance may be defined as the ratio of electrical voltage and current:

Z=V/I (1)

The impedance from tissue or biological material contains both resistive component and capacitive components due to the cell structure. It may be expressed as:

Z=R+jX=|Z|‰θ (2)

where Z is complex impedance, |Z| is magnitude, θ is phase angle, R is resistance and X is reactance. Impedance may also be expressed as, for example, resistance, capacitance, conductivity, relative permittivity, and combinations.

The impedance may be measured by sending a small current (e.g., <1 mA) to an electrode and sensing the current and the voltage to determine impedance. For example, as shown schematically in FIG. 2, a wire 200 having an electrode 202 adjacent its distal tip is introduced via catheter 204 into a vessel V. A signal source 206 provides a small AC current. A current sensor 208 and a voltage sensor 210 measure the current and voltage between the electrode 202 and a reference or ground electrode 212. Alternatively, the reference electrode could be carried on the wire 200. The impedance value may be calculated as the ratio of the two measurements. The voltage and current may be measured in both magnitude and phase angle so that complex impedance may be calculated.

When there is a large difference in contact area between the electrode 202 (which may be, for example 0.014″ in diameter) and the reference electrode (which may be for example 10 cm×20 cm), the material immediately surrounding the electrode contributes the most to the whole impedance. Thus the electrical impedance is more of a local measurement around the electrode. This local measurement may measure the tissue within about 3 or 4 times the diameter of the electrode 202.

When the electrode 202 is moved from one material to a different material, such as, for example, from blood to atheromatous plaque forming a partial or total chronic total occlusion (CTO), or from atheromatous plaque to the vessel wall, the measured impedance value will change significantly. This change in impedance can be used as a basis to guide the electrode 202, for example, to assure the vascular electrode remains in contact with the atheramatious. The electrode 202 can be controlled either by determining the nature of the material based upon its impedance, or by determining that the impedance has or has not changed and thus, the nature of the material in contact with the electrode is different or remains the same.

In one embodiment, at the beginning of a set of measurements, impedance may be measured with the electrode 202 in blood as a baseline measurement. The use of a baseline measurement may remove the contribution due to patient variation and measurement setup. Subsequent measurements may be made relative to the baseline.

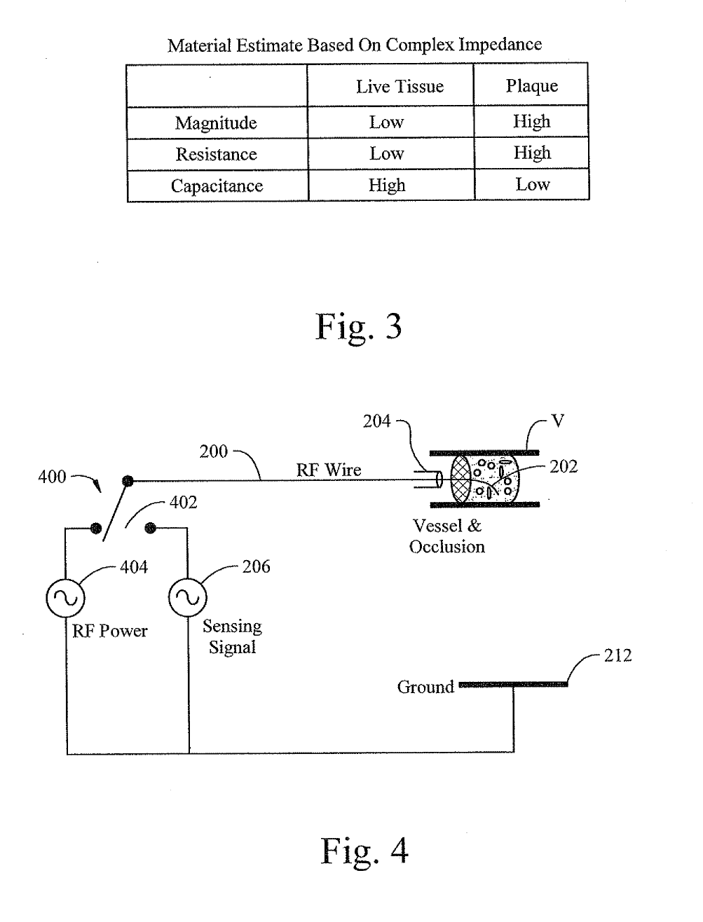

FIG. 3 lists example impedance change relative to a blood baseline measurement based upon tissue type. One may use these impedance components, alone or in combination, to identify or at least discriminate among tissue types.

The electrode 202 may be used to deliver RF power, as well as measure impedance. FIG. 4 shows an alternate example circuit topology 400 for alternating connection between impedance measurement and RF power delivery. The circuit 400 includes a switch 402, controlled by a manual or automated activation control, connects either impedance sensing signal source 206 or RF power source 404 to the electrode 202. The impedance measurement unit may be connected for the majority of the time. The RF power source 404 may be connected for very short periods (e.g. 1 to 3 seconds) for RF power delivery. Although no impedance is measured during RF activation, an operator may stop RF activations and use fluoroscopy to check the wire position before re-applying power.

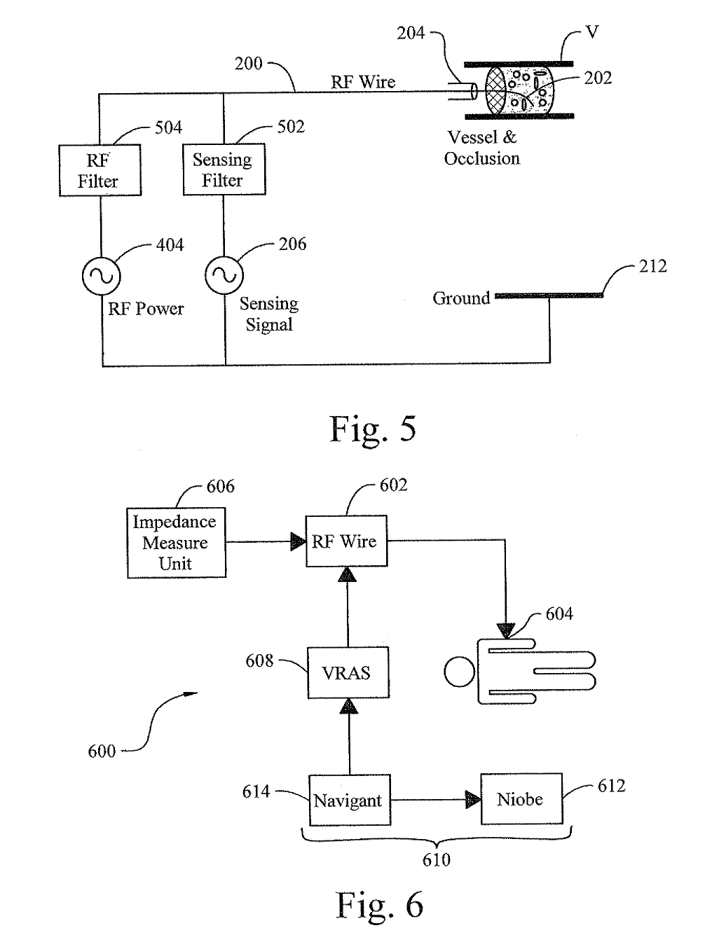

FIG. 5 shows an example circuit topology 500 for simultaneous impedance measurement and RF power delivery. For continuous configuration, RF power and impedance measurement signals are applied to the wire simultaneously from RF Power Source 404 and sensing signal source 206, but at different frequencies. Electrical filters 502 and 504 help prevent interference with the RF power and impedance sensing circuits.

The vascular probe may be used to measure multiple sites within a blood vessel. FIG. 6 is a schematic diagram showing a possible configuration of a system 600 for characterizing tissue within a blood vessel. The system comprises an elongate medical device 602, such as an RF wire, having at least one electrode 604 thereon. The system 600 further comprises an impedance measuring unit 606 connected to the elongate device 602 and electrode 604. A vascular remote advancement system 608 and a vascular electrode steering system 610 allow the elongate medical device to be advanced and oriented, respectively. In the preferred embodiment, the vascular electrode steering system 610 comprises a magnetic navigation system, such as a Niobe™ magnetic navigation system 612, available from Stereotaxis, Inc., St. Louis, Mo., under the control of a Navigant™ medical navigation interface 614, also available from Stereotaxis, Inc., St. Louis, Mo.

While as shown and described herein, the steering system is a magnetic steering system that applies a magnetic field of a selected direction on a magnetically responsive element carried on an elongate medical device, such an endoscope, catheter, or guidewire, the steering system may alternatively be based on, for example, mechanical, electrostriction, pneumatic, or hydraulic actuation technologies.

Under manual control of a physician, or automated control of a computer, the steering system 610 can move the electrode 604 in a circumferential manner to measure impedance at multiple sites within the blood vessel, such as, for example, 8 points at 45° apart, in a full circle around the circumference of the vessel. Once a circular measurement has been completed, the medical navigation interface 614 can actuate the VRAS 608 to advance or retract the elongate medical device 602 along a longitudinal axis of a blood vessel in a small increment or step, such as, for example, 1 mm, and upon completion of the advancement, and perform another series of measurements around the circumference of the vessel in the new location. This circular measurement of impedance values may be repeated for multiple steps of VRAS 604 movement, so that impedance can be measured along a section of the blood vessel. In a similar fashion, measurements may be made in a spiral pattern of locations along a section of the blood vessel.

By localizing the electrode where the measurements are made, the impedance values (e.g. Z, R and X) can be projected on a display (not shown) such as an impedance map (e.g., a virtual tube representing the blood vessel). Based on the impedance map, one can easily locate an atheromatous plaque spot in the vessel using the impedance values, such as an area of high resistance or low capacitance, as described above. These points can be distinctively displayed by color or intensity (brightness) or constancy (e.g. flashing). The type of material encountered in the blood vessel can be identified based upon the measured impedances (either the absolute or relative values) as described above, and each type of material distinctively displayed.

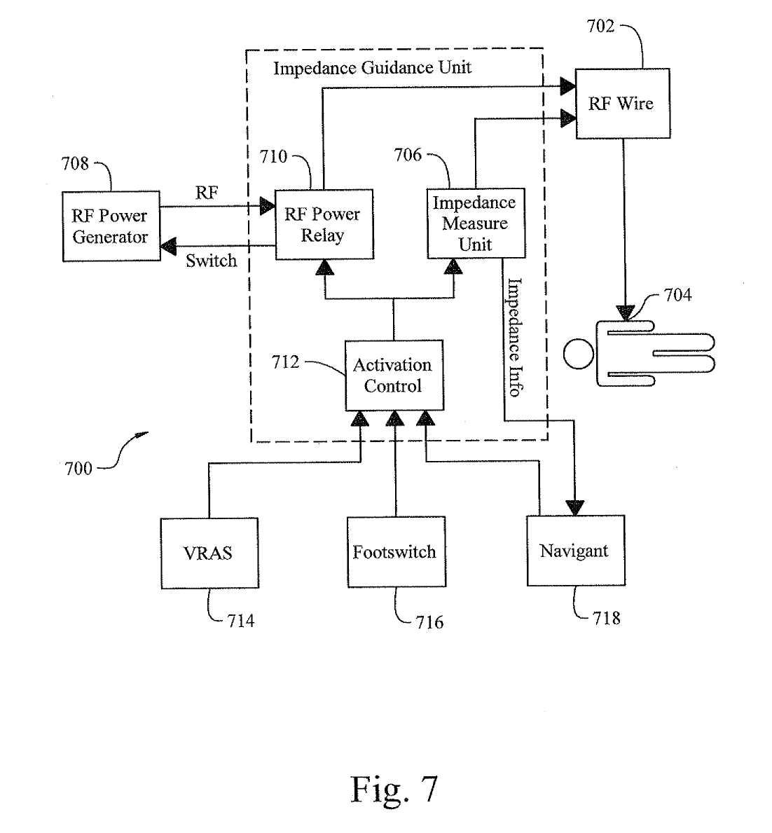

Impedance measurements may be used to guide a vascular electrode through a partial or total vascular occlusion. FIG. 7 shows an example system 700 for RF occlusion canalization. The system comprises an elongate medical device, such as an RF wire 702 having at least one electrode 704 thereon. An impedance measuring unit 706, an RF power generator 708, an RF power relay 710, an activation control 712, a VRAS 714, a footswitch 716, and a RF wire steering system, such as a magnetic steering system, such as a Navigant™ system 718 controlling a magnetic navigation system (not shown), such as a Niobe™ magnet system which creates a magnetic field in a selected direction to orient a magnetically responsive element on the RF wire 702.

In operation, the steering system, the Navigant system 718 and VRAS 710 are actuated to place the vascular electrode into contact with the atheromatous plaque in the vessel. When the impedance measurements indicate that the electrode is in contact with atheromatous plaque, the physician activates the footswitch 716 to signal the application of RF power to ablate the atheromatous plaque. The activation control 712 measures the impedance at electrode 704 to verify that the electrode 704 is contacting atheromatous plaque and not vessel wall. If the impedance indicates that the electrode 704 is contacting atheromatous plaque, then the activation control 712 again switches on the RF power relay 710 to send RF power from the RF power generator 708 to the vascular electrode to ablate the atheromatous plaque.

The impedance information is sent to the steering system so the electrode 704 is maintained within the atheromatous plaque. By incrementally advancing the electrode 704, vaporizing the atheromatous plaque, and advancing the electrode 704 to contact additional atheromatous plaque the system re-canalizes the vessel.

One method of guiding an electrode through a vascular occlusion includes contacting tissue at a first location in the vessel with the vascular electrode, measuring a first impedance between the vascular electrode and a reference electrode, moving the vascular electrode to contact tissue at a second location in the vessel, and measuring a second impedance between the vascular electrode and the reference electrode. The measured impedance at each location may be sufficient to determine the nature of the tissue at each location, or the difference between the measured impedance at each location may be sufficient to determine the nature of the tissue at each location. In this manner, it generally can be determined whether the vascular electrode is in contact with healthy tissue (of relatively low impedance), or with athromotous material (of relatively high impedance). When it has been reliably determined that the electrode is in contact with athromotous material, RF energy can be supplied to the vascular electrode to ablate the athromotous material. On the other hand, if it is determined that the electrode is not in contact with athromotous material, the electrode can be moved to a different location, where the impedance can again be measured to determine the nature of the surrounding tissue.

Example embodiments are provided so that this disclosure will be thorough, and will fully convey the scope to those who are skilled in the art. Numerous specific details are set forth, such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of guiding a vascular electrode through a vascular occlusion comprising:
contacting the vascular occlusion with the vascular electrode;
measuring a first impedance between the vascular electrode and a reference electrode;
moving the vascular electrode;
measuring a second impedance between the vascular electrode and the reference electrode; and
if the difference in impedance between the first measured impedance and the second measured impedance is large enough to indicate that the vascular electrode is no longer contacting the vascular occlusion, then moving the vascular electrode until the vascular electrode contacts the vascular occlusion.

2. The method of claim 1 further comprising energizing the vascular electrode with radio frequency electricity sufficient to vaporize part of the vascular occlusion.

3. The method of claim 2 further comprising energizing the vascular electrode with radio frequency electricity if the second measured impedance indicates that the vascular electrode is in contact with the vascular occlusion.

4. The method of claim 1 wherein the second measured impedance is a measurement of resistivity.

5. The method of claim 1 wherein the second measured impedance is a measurement of relative permittivity.

6. The method of claim 1 wherein the second measured impedance is a measurement of complex impedance.

7. The method of claim 1 wherein the second measured impedance is a measurement relative to a baseline impedance measured in blood.

8. The method of claim 1 wherein the second measured impedance is measured by energizing the vascular electrode with an excitation voltage of between 20 kHz and 500 kHz.

9. A system for radio frequency (RF) wire guidance during RF occlusion canalization comprising:
a RF wire configured to travel through a blood vessel;
an impedance measure unit configured to measure an electrical impedance between the RF wire and a reference electrode;
a RF power source;
an activation controller configured to connect the RF power source to the RF wire when the impedance measure unit indicates that the RF wire is contacting an occlusion material;
a RF wire steering system; and
a controller configured to activate the steering system to maintain the RF wire within the occlusion material based on impedance measured by the impedance measure unit.

10. The system of claim 9 wherein the activation controller is further configured to alternate connecting the RF power source and the impedance measure unit to the RF wire.

11. The system of claim 9 wherein the RF power source and the impedance measure unit are simultaneously connected to the RF wire.

12. A method of characterizing tissue within a blood vessel comprising:
contacting a first blood vessel site with a vascular electrode;
measuring the impedance between the vascular electrode and a reference electrode;
moving the vascular electrode to a second blood vessel site;
repeating the measuring and the moving until the impedance of a plurality of vessel sites have been measured; and
identifying the material type of the blood vessel sites based on the measured impedances.

13. The method of claim 12 further comprising displaying the measured impedances on a display as an impedance map.

14. The method of claim 12 wherein the measurements are relative to a baseline impedance measured in blood.

15. The method of claim 12 wherein the measurements measure resistivity.

16. The method of claim 12 wherein the measurements measure relative permittivity.

17. The method of claim 12 wherein the measurements measure complex impedance.

18. The method of claim 12 wherein the measurements are measured by energizing the vascular electrode with an excitation voltage of between 20 kHz and 500 kHz.

19. The method of claim 12 wherein a subset of the plurality of vessel sites forms a ring around an inner circumference of the blood vessel.

20. The method of claim 12 wherein multiple subsets of the plurality of vessel sites form a plurality of rings around inner circumferences of the blood vessel.

21. The method of claim 12 wherein a subset of the plurality of vessel sites forms a spiral along an inner surface of the blood vessel.

22. A system comprising:
a vascular electrode;
an impedance measure unit connected to the vascular electrode;
a vascular remote advancement system configured to advance or retract the vascular electrode along a longitudinal axis of a blood vessel;
a steering system configured to steer the vascular electrode within the blood vessel; and
a controller configured to:
actuate the vascular remote advancement system and the steering system to contact the vascular electrode to a plurality of blood vessel sites; and
measure the impedance of the plurality of blood vessel sites with the impedance measure unit.

23. A system configured to perform the method of claim 1.

24. A system configured to perform the method of claim 12.
